(19) United States
(12) Patent Application Publication
(10) Pub. No.: US 2012/0197168 A1
(43) Pub. Date: Aug. 2, 2012

(54) PELVIC ORTHOSIS SYSTEMS AND METHODS

(75) Inventors: Sunil K. Agrawal, Newark, DE (US); Mustafa Shabbir Kurbanhusen, Pittsburgh, PA (US); Vineet Vashista, Newark, DE (US)

(73) Assignee: University of Delaware, Newark, DE (US)

(21) Appl. No.: 13/358,959

(22) Filed: Jan. 26, 2012

Related U.S. Application Data
(60) Provisional application No. 61/437,289, filed on Jan. 28, 2011.

(51) Int. Cl. A61F 5/00

(52) U.S. Cl. 602/19

(57) ABSTRACT

Pelvic orthosis systems and methods are disclosed. An orthosis system comprises a frame for supporting the orthosis system, a hip brace adapted to secured to a hip of a user, and a plurality of elastic members extending from the hip brace to the frame. An orthosis method comprises securing a user to an orthosis system having a frame, a hip brace, and a plurality of elastic members extending from the hip brace to the frame, and enabling the user to walk while secured to the orthosis system.

### CROSS REFERENCE TO RELATED APPLICATIONS

This application claims priority to U.S. Patent Application No. 61/437,289, entitled “TETHERED PELVIC ASSIST DEVICE (T-PAD) FOR CONTROLLED TRAINING OF HUMAN GAIT,” filed on Jan. 28, 2011, the contents of which are incorporated herein by reference in their entirety.

### FIELD OF THE INVENTION

The present invention relates generally to orthosis systems and methods, and more particularly, to pelvic orthosis systems and methods for use in assisting walking.

### BACKGROUND OF THE INVENTION

Often, patients with significant one-time or permanent illnesses that affect the neurological system (e.g. stroke, traumatic spinal cord injury, cerebral palsy, spina bifida) will experience degradation in their control of motor skills. In particular, this degradation in control can significantly affect a patient's walking ability. Thus, patients may require physical therapy following a neurological illness to regain walking ability.

A number of orthosis (or orthotic) systems have been developed to assist patients in recovering their ability to walk. One such orthosis system is disclosed in U.S. patent application Ser. No. 11/113,729, entitled “GRAVITY BALANCED ORTHOSIS APPARATUS,” filed on Apr. 25, 2005, the contents of which are incorporated herein by reference in their entirety. These systems operate by retraining the coordination among the joints within and across a user's impaired limbs during a walking motion. The coordination and motion of the user's pelvis is particularly important during this walking motion. By repetitively practicing with an orthosis system, the user may redevelop their motor skills and recover walking ability. However, time spent during this rehabilitation process may be frustrating or painful for the patient. Accordingly, orthosis systems that improve recovery time and patient comfort are desired.

### SUMMARY OF THE INVENTION

Aspects of the present invention relate to pelvic orthosis systems and methods.

In accordance with one aspect of the present invention, an orthosis system is disclosed. The orthosis system comprises a frame for supporting the orthosis system, a hip brace adapted to secured to a hip of a user, and a plurality of elastic members extending from the hip brace to the frame.

In accordance with another aspect of the present invention, an orthosis method is disclosed. The orthosis method comprises securing a user to an orthosis system having a frame, a hip brace, and a plurality of elastic members extending from the hip brace to the frame, and enabling the user to walk while secured to the orthosis system.

### BRIEF DESCRIPTION OF THE DRAWINGS

The invention is best understood from the following detailed description when read in connection with the accompanying drawings, with like elements having the same reference numerals. When a plurality of similar elements are present, a single reference numeral may be assigned to the plurality of similar elements with a small letter designation referring to specific elements. When referring to the elements collectively or to a non-specific one or more of the elements, the small letter designation may be dropped. According to common practice, the various features of the drawings are not drawn to scale unless otherwise indicated. To the contrary, the dimensions of the various features may be expanded or reduced for clarity. Included in the drawings are the following figures:

FIGS. 1A-1D illustrate an exemplary orthosis system in accordance with aspects of the present invention;

FIGS. 2-5 illustrate exemplary configurations of the orthosis system of FIGS. 1A-1D;

FIG. 6 illustrates an exemplary configuration for the markers of the orthosis system of FIGS. 1A-1D; and

FIG. 7 is an exemplary orthosis method in accordance with aspects of the present invention.

### DETAILED DESCRIPTION OF THE INVENTION

The orthosis systems and methods disclosed herein are usable to assist a user in developing or regaining the ability to walk. Generally, these systems and methods generate forces for supporting the user's pelvis and assisting the user in swinging his or her legs in a normal walking motion. The walking motion is desirably performed under the user's own power. The systems and methods may be used by users who are performing walking motions on a treadmill.

The various aspects of the present invention relate generally to pelvic orthosis systems adapted to be secured to the hip of a user for guiding the motion of the user. The orthosis system has a hip brace and a plurality of elastic members extending from the hip brace. The elastic members extend to various attachment points positioned on a frame surrounding the hip brace. The layout and tension of the elastic members is specifically selected to assist a user in performing a walking motion.

The systems and methods disclosed herein are particularly suitable for users that are developing or redeveloping control of motor skills due to neurological disorders including, for example, stroke, traumatic spinal cord injury, cerebral palsy, or spina bifida. These systems and methods may be effective for both adults and children.

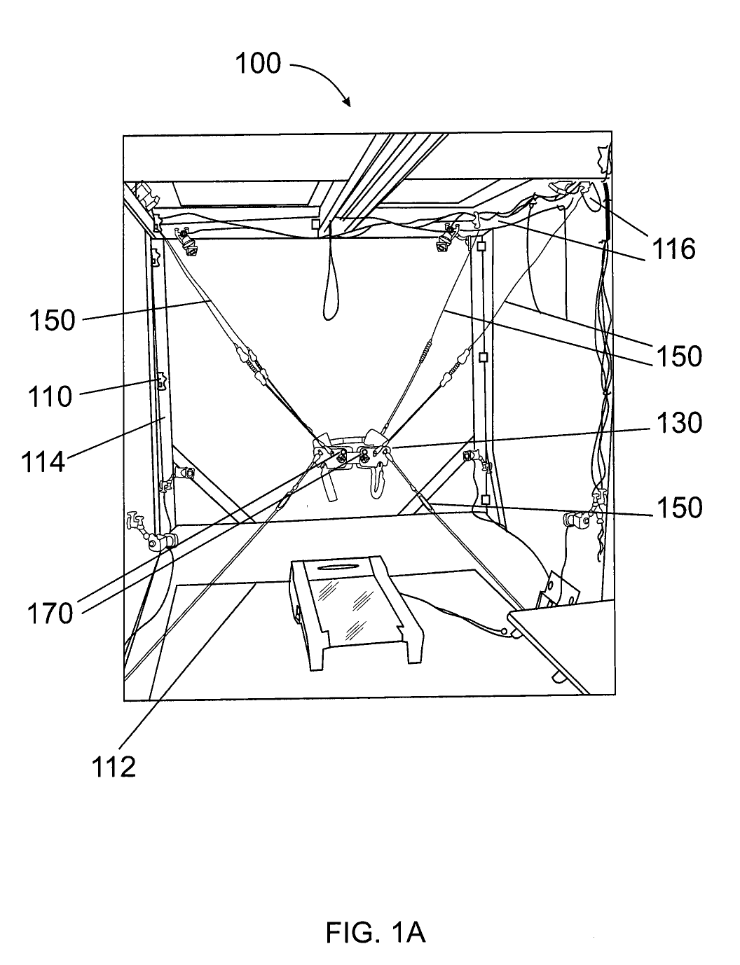

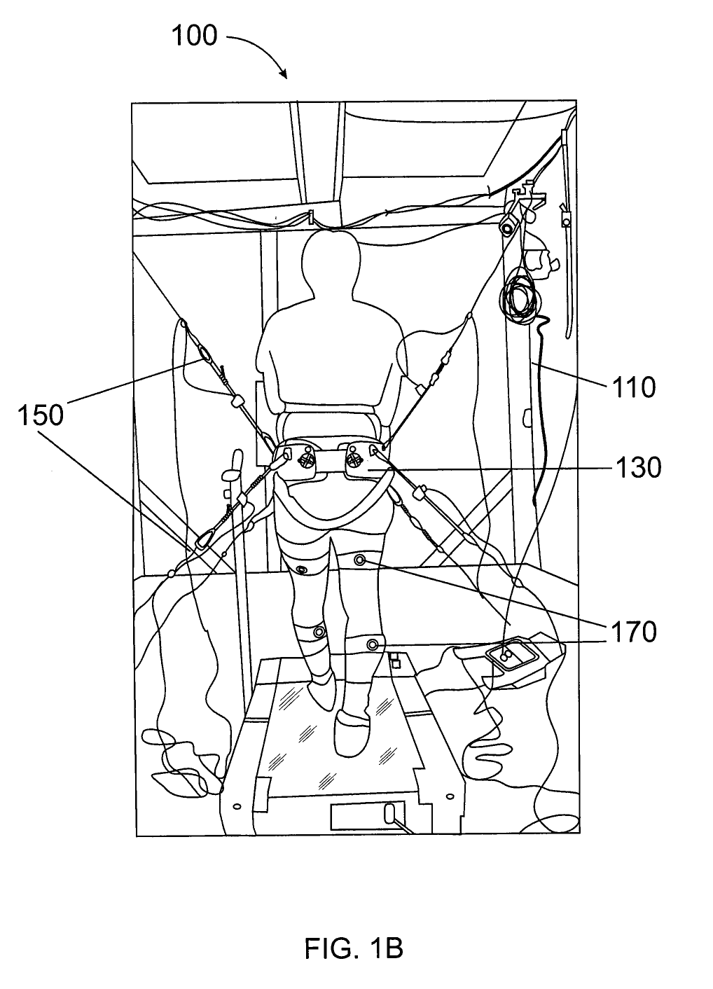

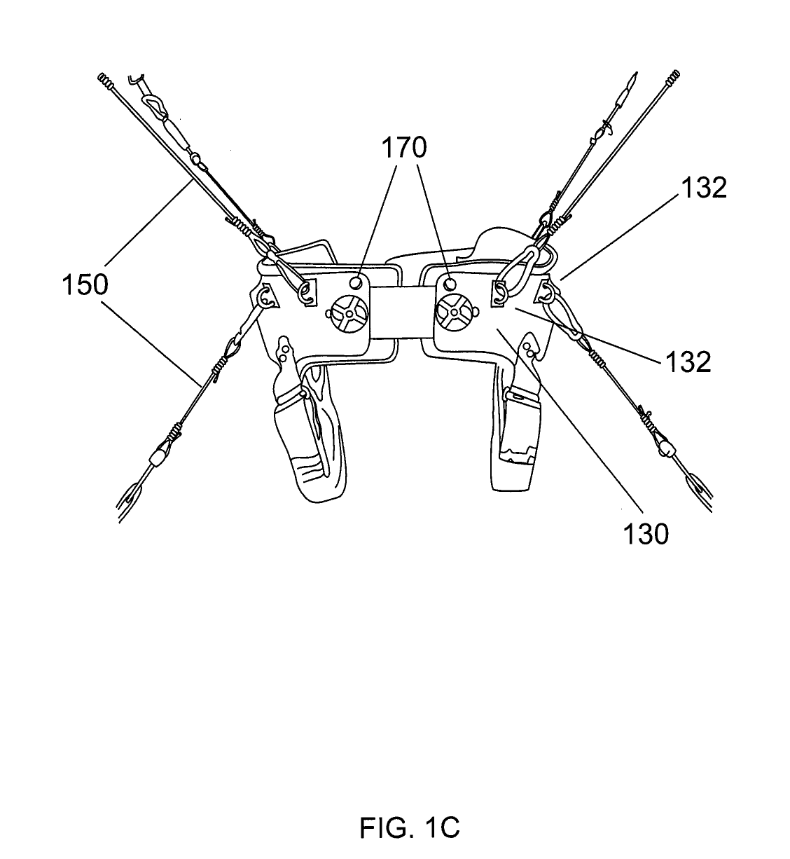

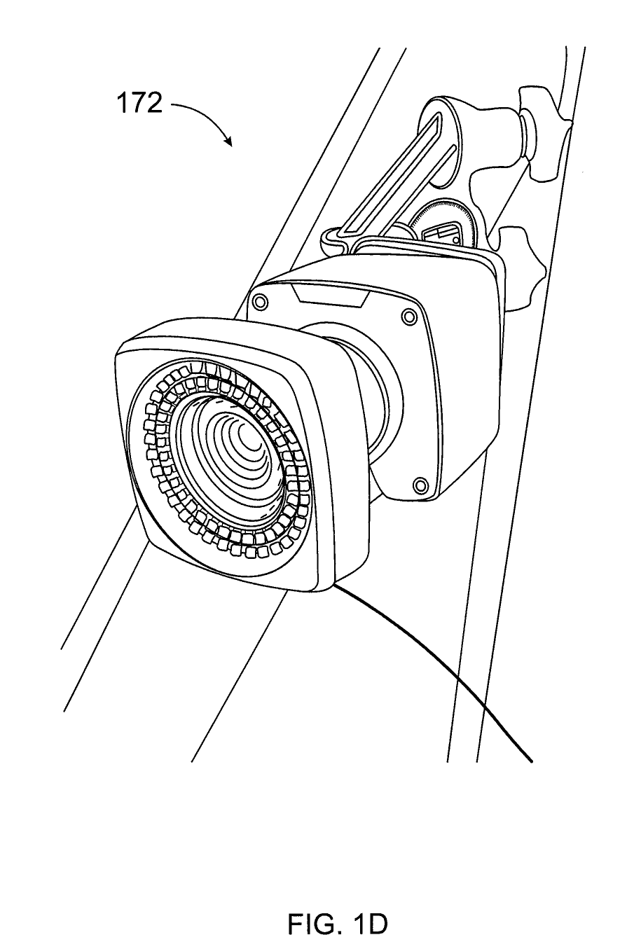

Referring now to the drawings, FIGS. 1A-1D illustrate an exemplary orthosis system 100 in accordance with aspects of the present invention. Orthosis system 100 may be usable to assist a user in developing or regaining the ability to walk. As a general overview, orthosis system 100 includes a frame 110, a hip brace 130, and a plurality of elastic members 150. Additional details of orthosis system 100 are described herein.

Frame 110 supports orthosis system 100. As used herein, the term “frame” is intended to encompass any and all structures capable of supporting the orthosis system. The term “frame” is not intended to be limited to any free-standing structure specifically dedicated for use with the orthosis system. For example, and without limitation, a frame may include a free-standing movable or non-movable rigid structure having a base portion and an upright portion. For another example, a frame may include walls, ceilings, floors, or other structures integral to a building in which the orthosis system is positioned and operated. The frame may, but need not, surround the user one or more sides, and extend above and below the user's pelvis. Suitable structures for use as a frame of the present invention will be understood to one of ordinary skill in the art from the description herein.

In an exemplary embodiment, frame 110 includes a base portion 112 and an upright portion 114, as shown in FIG. 1A. Base portion 112 may be placed on a walking surface. Base portion 112 is shaped to allow room for a user of orthosis system 100 to perform a walking motion. In a preferred embodiment, base portion 112 is shaped to accommodate a treadmill on which the user performs the walking motion. Base portion 112 of frame 110 may include wheels to enable movement of orthosis system 100. Upright portion 114 extends in an upright direction from base portion 112. Upright portion 114 includes a plurality of attachment points 116. Each attachment point 116 is configured to receive and secure one end of one of the elastic members 150, as will be described in greater detail below. In an exemplary embodiment, attachment points 116 comprise U-bolts provided on frame 110, with elastic members 150 including carabiners to secure elastic members 150 to the U-bolts. Frame 110 may be fashioned from any suitable rigid material using conventional manufacturing processes.

Hip brace 130 is adapted to be secured to the hip of a user. As used herein, the term “brace” is intended to encompass any and all structures adapted to be secured or coupled to a portion of the user of the orthosis system. For example, and without limitation, the braces of the present invention may include and/or refer to straps, belts, buckles, fasteners, garments, or any other structure adapted for attachment to the user. The braces may, but need not, include structures adapted to support, align, or otherwise hold part of the user in a certain position or angle. Suitable structures for use as braces of the present invention will be understood to one of ordinary skill in the art from the description herein.

Hip brace 130 anchors the user of orthosis system 100 to frame 110. In an exemplary embodiment, hip brace 130 comprises a padded belt adapted to be secured around the hips of the user, as shown in FIGS. 1A and 1B. Hip brace 130 may be secured in place using suitable fasteners such as, for example, a buckles or straps. Hip brace 130 also includes a plurality of attachment points 132, as shown in FIG. 1C. Each attachment point 132 is configured to receive and secure the other end of one of the elastic members 150, as will be described in greater detail below. Attachment points 132 may comprise any of the structures described above with respect to attachment points 116. Suitable hip braces 130 for use in the present invention will be understood by one of ordinary skill in the art from the description herein.

Elastic members 150 extend from and couple hip brace 130 to frame 110. Elastic members 150 are attached at one end to attachment points 132 on hip brace 130, and are attached at the other end to attachment points 116 on frame 110. Elastic members 150 exert a force on hip brace 130 depending on the tension of elastic members 150 and the direction in which elastic members 150 extend from hip brace 130 toward frame 110. The force provided by elastic members 150 is useful to support the user's pelvis and assist the user in swinging his or her legs in a normal walking motion.

In an exemplary embodiment, each elastic member 150 comprises at least one metal spring, as shown in FIG. 1A. Suitable springs include, for example, one or more steel wired extension springs. The steel wired springs may have stiffness levels, for example, of 13 lb/in, 18 lb/in and/or 24 lb/in. Elastic members 150 may comprise multiple springs connected either in parallel or in series, to obtain the desired tension. Additionally, elastic members 150 may comprise a combination of cables and springs connected between the respective attachment points 116 and 132. Again, the cables and springs may be connected either in parallel or in series, to obtain the desired tension. One or more carabiners may be used to releasably attached the springs to other springs, cables, or directly to attachment points 116 and 132.

The force exerted on hip brace 130 by elastic members 150 is dependent at least in part on the tension of elastic members 150. The tension in elastic members 150 may be an innate property of each elastic member 150, determined, for example, by the spring constant of a spring of the elastic member 150. In a preferred embodiment, the tension in at least one elastic member 150 is adjustable. This may be desirable in order to adjust the forces provided by elastic members 150, to tailor the forces exerted on hip brace 130 to the support and assistance needs of a particular user of orthosis system 100. Suitable structures for adjusting the tension of elastic members 150 include, for example, tie-downs or ratchets for increasing and decreasing the length of elastic members 150. In another preferred embodiment, orthosis system 100 may include a plurality of elastic members 150 that each have different tensions. In this embodiment, elastic members 150 may be freely removable from orthosis system 100 and replaceable with other elastic members 150 having different tensions, to tailor the forces exerted on hip brace 130 to the support and assistance needs of a particular user of orthosis system 100.

The force exerted on hip brace 130 by elastic members 150 is also dependent at least in part on the direction in which elastic members 150 extend from hip brace 130 toward frame 110. A number of different configurations of elastic members 150 are envisioned in accordance with aspects of the present invention. The configurations may differ from each other based on the number of elastic members 150, the tension variations in each elastic member 150, and the directions in which each elastic member 150 extends. Selected ones of these configurations will be described herein in accordance with aspects of the present invention. However, it will be understood by one of ordinary skill in the art that the configurations described herein are for the purposes of illustration, and are not intended to be limiting.

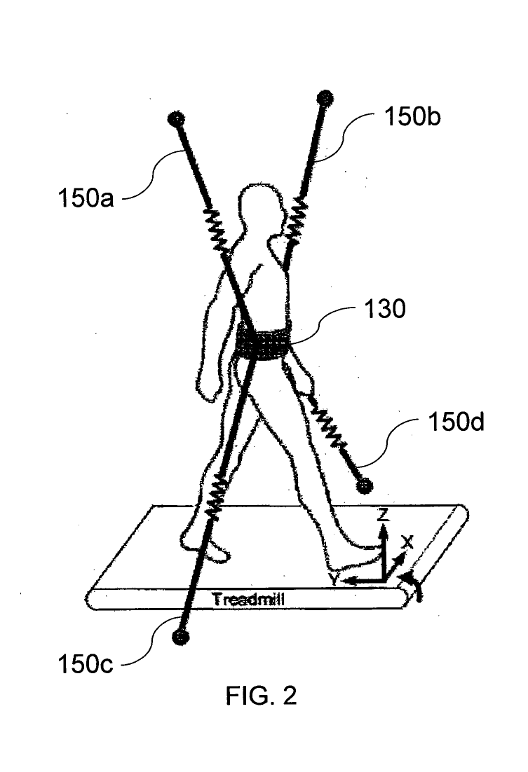

FIGS. 2-5 illustrate exemplary configurations for orthosis system 100 in accordance with aspects of the present invention. As shown in FIGS. 2-5, the configurations generally include two elastic members 150a and 150b extending from hip brace 130 to a portion of frame 110 positioned above hip brace 130 (when hip brace 130 is secured to a user). Elastic members 150a and 150b may further extend to a portion of frame 110 positioned above a user's head. These configurations also includes two elastic members 150c and 150d extending from hip brace 130 to a portion of frame 110 positioned below hip brace 130 (when hip brace 130 is secured to a user). Elastic members 150c and 150d may further extend to a portion of frame 110 positioned below a user's feet.

Elastic members 150a, 150b, 150c, and 150d may all extend in different planar directions relative to each other. However, in a preferred embodiment (shown in FIG. 2), elastic members 150a, 150b, 150c, and 150d extend in substantially the same plane (i.e. the XZ plane in FIG. 2). In a more preferred embodiment, elastic members 150a, 150b, 150c, and 150d extend in the plane that is substantially orthogonal to the walking direction of the user of orthosis system 100 (i.e. the Y direction in FIG. 2). This configuration may be particularly beneficial for assisting a user of orthosis system 100 in performing a walking motion, because this configuration provides partial constraint to the user's pelvis, i.e., only in the coronal plane.

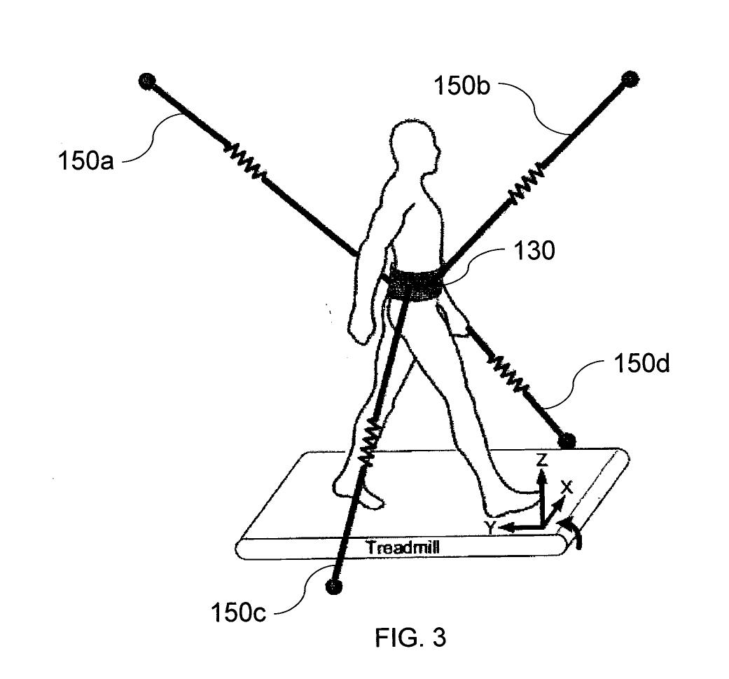

In another preferred embodiment (shown in FIG. 3), elastic members 150a and 150b extend in substantially one plane (i.e. the YZ plane in FIG. 3), and elastic members 150c and 150d extend in substantially another plane (i.e. the XZ plane in FIG. 3). In a more preferred embodiment, elastic members 150a and 150b extend in the plane that is substantially parallel to the walking direction of the user of orthosis system 100, and elastic members 150c and 150d extend in the plane that is substantially orthogonal to the walking direction of the user of orthosis system 100 (i.e. the Y direction in FIG. 3). This configuration may also be particularly beneficial for assisting a user of orthosis system 100 in performing a walking motion, because this configuration provides asymmetric forces on the user's pelvis. This particular configuration may be used to provide counter-clockwise torque on the pelvis (viewed from the top of frame 110). This configuration may be used to simulate asymmetricity in healthy humans and resolve asymmetric gait problems in patients.

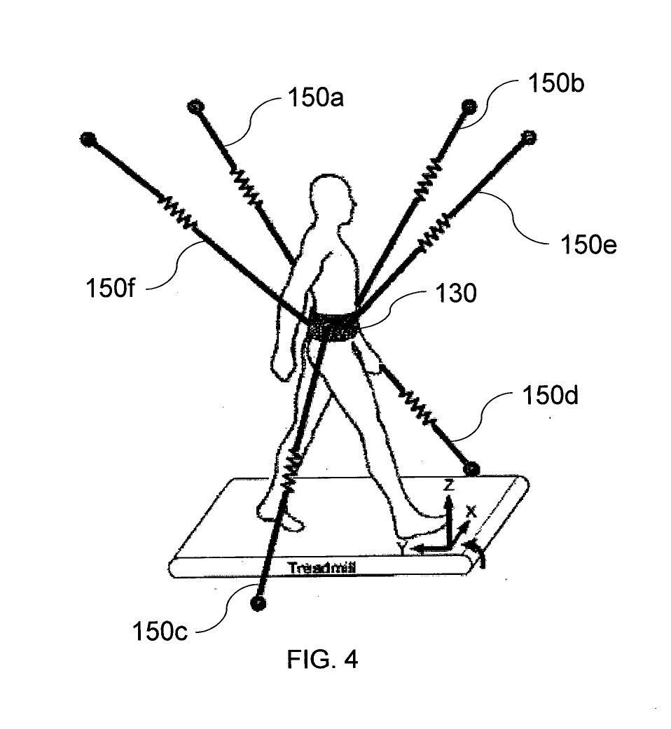

In yet another preferred embodiment (shown in FIG. 4), orthosis system 100 includes additional elastic members 150e and 150f. Elastic members 150e and 150f extend from hip brace 130 to a portion of frame 110 positioned above hip brace 130. In this embodiment, elastic members 150a and 150e extend in substantially one plane, elastic members 150c and 150d extend in substantially another plane, and elastic members 150b and 150f extend in substantially a third plane. In a more preferred embodiment, elastic members 150a and 150e extend in the plane that travels from a point forward and right of a user to a point rearward and left of the user; elastic members 150c and 150d extend in the plane that is substantially orthogonal to the walking direction of the user of orthosis system 100; and elastic members 150b and 150f extend in the plane that travels from a point forward and left of a user to a point rearward and right of the user. This configuration may also be particularly beneficial for assisting a user of orthosis system 100 in performing a walking motion, because this configuration provides full constraint to the pelvis and can lift and partially or completely support a user's weight.

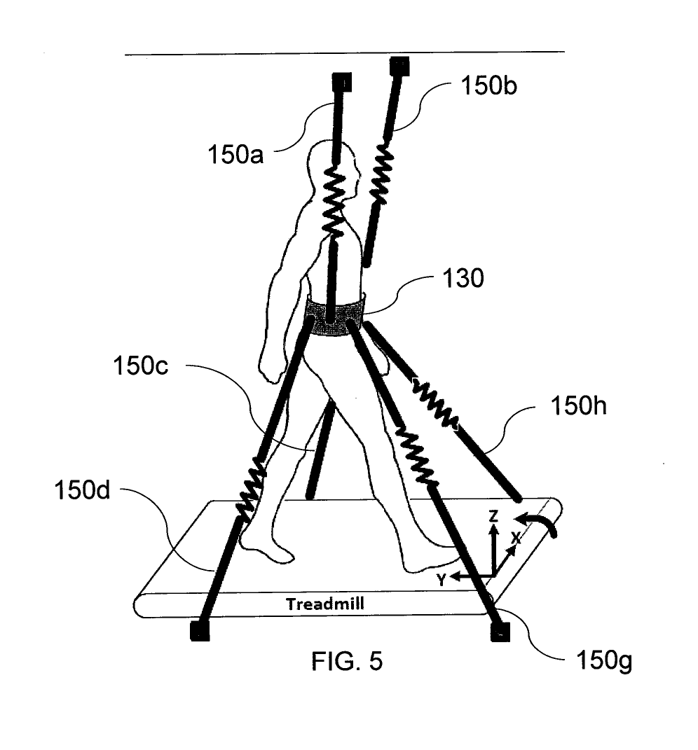

In yet another preferred embodiment (shown in FIG. 5), orthosis system 100 includes additional elastic members 150g and 150h. Elastic members 150g and 150h extend from hip brace 130 to a portion of frame 110 positioned below hip brace 130. In this embodiment, elastic members 150a and 150b extend in substantially one plane, elastic members 150c and 150g extend in substantially another plane, and elastic members 150d and 150h extend in substantially a third plane. In a more preferred embodiment, elastic members 150a and 150b extend in the plane that is substantially orthogonal to the walking direction of the user of orthosis system 100; elastic members 150c and 150g extend in the plane that travels from a point forward and right of a user to a point rearward and left of the user; and elastic members 150d and 150h extend in the plane that travels from a point forward and left of a user to a point rearward and right of the user. This configuration may also be particularly beneficial for assisting a user of orthosis system 100 in performing a walking motion, because this configuration adds on to the user's weight by applying downward forces on the pelvis, while still retaining symmetry and stability of the user.

Orthosis system 100 is not limited to the above components, but may include alternative or additional components, as would be understood by one of ordinary skill in the art.

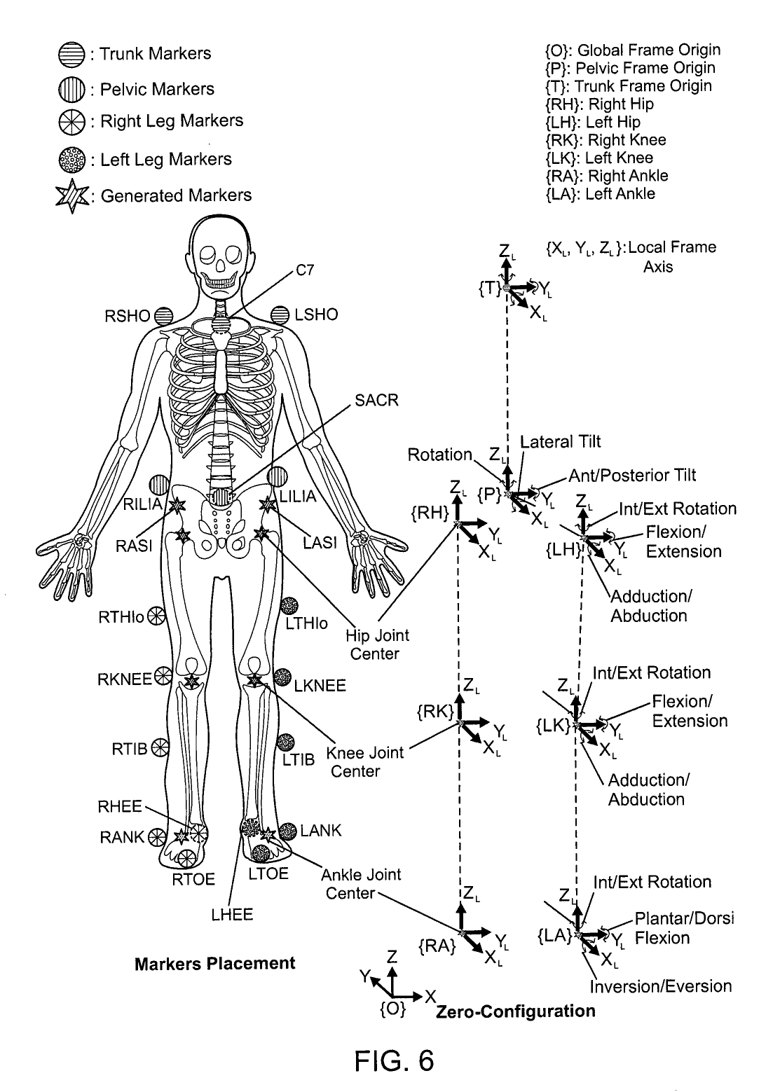

For one example, orthosis system 100 may include a motion capture system. The motion capture system is oriented to record a walking motion of the user when the user is secured to hip brace 130. In an exemplary embodiment, as shown in FIG. 1A-1C, the motion capture system comprises a plurality of markers 170 adapted to be secured to one or both of hip brace 130 and the user of orthosis system 100. As shown in FIG. 1D, the motion capture system further comprises a video camera 172 configured to record data representing movement of the markers 170 during a walking motion of the user. Suitable motion capture systems for use with the present invention include the BONITA family of systems provided by Vicon Motion Systens, Ltd., of Oxford, United Kingdom.

Where markers 170 are attached to the user of orthosis system 100, their positioning may be important for properly tracking and storing the walking motion of the user during use. FIG. 6 illustrates an exemplary layout for markers 170 on the leg of a user in accordance with aspects of the present invention. The use of the layout in FIG. 6 may be particularly desirable for accurately capturing the user's movements while secured to orthosis system 100.

The motion capture system may further include a processor for processing the movement data collected by video camera 172. This may be desirable in order to analyze the user's walking motion to determine changes that are produced or desired. For example, the motion capture system may record the user's walking motion while they are unsecured and while they are secured to hip brace 130, in order to identify changes in the user's walking motion. The motion capture system may further include a memory for storing the recorded and/or processed data. Suitable processing and memory components will be known to one of ordinary skill in the art from the description herein. In a preferred embodiment, the analyzed information may be used to adjust the number, position, and tension of elastic members 150, in order to optimize the user's walking motion.

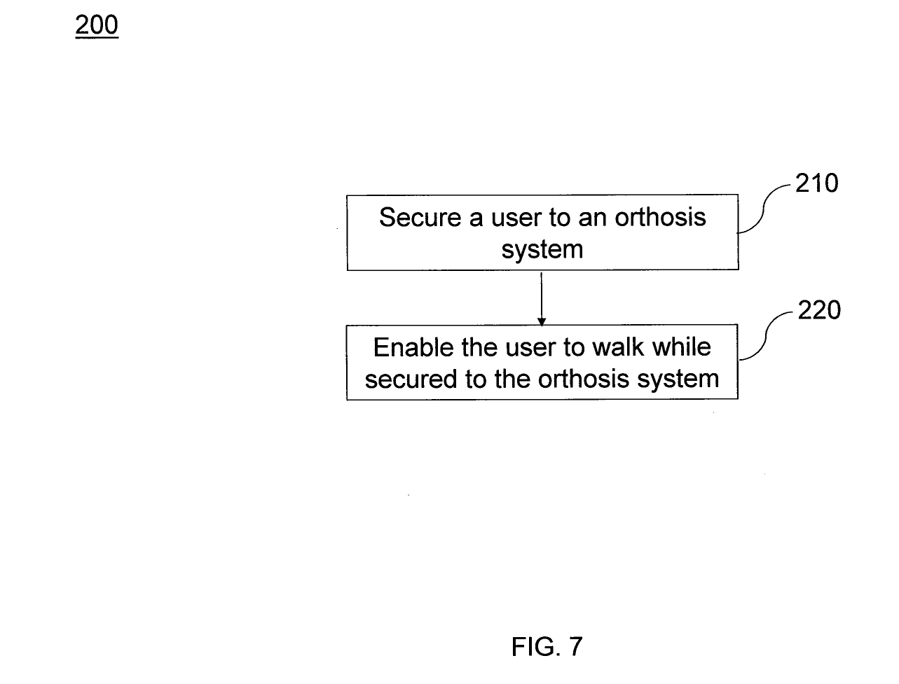

FIG. 7 shows an exemplary orthosis method 200 in accordance with aspects of the present invention. Method 200 may be implemented to assist a user in developing or regaining the ability to walk. As a general overview, method 200 includes securing a user to an orthosis system, and enabling the user to walk. Additional details of method 200 are described herein with respect to the components of orthosis system 100.

In step 210, a user is secured to an orthosis system. In an exemplary embodiment, the user is secured to orthosis system 100. Hip brace 130 is secured around the hips of the user. Elastic members 150 are connected such that they extend from attachment points 132 on hip brace 130 to attachment points 116 on frame 110. Step 210 may further comprise adjusting the tension of one or more elastic members 150 once they are connected. As set forth above, this may be desirable in order to adjust the forces provided by elastic members 150, to tailor the forces exerted on hip brace 130 to the support and assistance needs of a particular user of orthosis system 100.

In step 220, the user is enabled to walk. In an exemplary embodiment, the user walks while secured to orthosis system 100. As set forth above, the user may desirably be positioned on a treadmill to enable the user to perform a walking motion without requiring movement of orthosis system 100. As the user performs the walking motion, orthosis system 100 generates forces for supporting the user's pelvis and assisting the user in swinging his or her legs in a normal walking motion.

Method 200 is not limited to the above steps, but may include alternative steps and additional steps, as would be understood by one of ordinary skill in the art from the description herein.

For one example, it may be desirable to record the use of the orthosis system in order to monitor and assist in the progress of the user in developing or regaining the ability to walk. Accordingly, method 200 may further include the step of recording a walking motion of the user with a motion capture system while the user is secured to the orthosis system. In an exemplary embodiment, a plurality of markers 170 are secured to one of both of hip brace 130 and the user of orthosis system 100. The walking motion of the user is then recorded with a video camera 172 configured to record data representing movement of the markers 170.

As set forth above, the recorded data may further be processed and stored, as desired, in order to analyze the user's walking motion to determine changes that are produced or desired. The number, positioned, and tension of elastic members 150 may then be adjusted based on the recorded or processed data, in order to optimize the user's walking motion.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. An orthosis system comprising:
a frame for supporting the orthosis system;
a hip brace adapted to secured to a hip of a user; and
a plurality of elastic members extending from the hip brace to the frame.

2. The orthosis system of claim 1, wherein a tension of at least one of the plurality of elastic members is adjustable.

3. The orthosis system of claim 1, where a tension of at least one of the plurality of elastic members is different from a tension of at least another one of the plurality of elastic members.

4. The orthosis system of claim 1, wherein the plurality of elastic members comprises:
a first elastic member extending from the hip brace to a portion of the frame positioned above the hip brace; and
a second elastic member extending from the hip brace to a portion of the frame positioned below the hip brace.

5. The orthosis system of claim 4, wherein the plurality of elastic members further comprises:
a third elastic member extending from the hip brace to the portion of the frame positioned above the hip brace; and
a fourth elastic member extending from the hip brace to the portion of the frame positioned below the hip brace.

6. The orthosis system of claim 5, wherein the first, second, third, and fourth elastic members extend in substantially a same plane.

7. The orthosis system of claim 6, wherein the plane is substantially orthogonal to a direction of walking of the user.

8. The orthosis system of claim 5, wherein the first and third elastic members extend in substantially a same first plane, and the second and fourth elastic members extend in substantially a same second plane different from the first plane.

9. The orthosis system of claim 8, wherein the first plane is substantially parallel to a direction of walking of the user, and the second plane is substantially orthogonal to the direction of walking of the user.

10. The orthosis system of claim 5, wherein the plurality of elastic members further comprises:
fifth and sixth elastic member extending from the hip brace to the portion of the frame positioned above the hip brace.

11. The orthosis system of claim 1, further comprising a motion capture system oriented to record a walking motion of the user when the user is secured to the hip brace.

12. The orthosis system of claim 11, wherein the motion capture system comprises:
a plurality of markers adapted to be secured to one or both of the user and the hip brace; and
a video camera configured to record data representing a movement of the markers during the walking motion of the user.

13. The orthosis system of claim 12, wherein the motion capture system further comprises:
a processor adapted to process the data representing the movement of the markers; and
a memory adapted to store the processed data.

14. An orthosis method comprising:
securing a user to an orthosis system having a frame, a hip brace, and a plurality of elastic members extending from the hip brace to the frame; and
enabling the user to walk while secured to the orthosis system.

15. The orthosis method of claim 14, further comprising the steps of:
adjusting a tension of at least one of the plurality of elastic members.

16. The orthosis method of claim 14, further comprising the step of:
recording a walking motion of the user with a motion capture system while the user is secured to the orthosis system.

17. The orthosis system of claim 16, wherein the recording step comprises:
securing a plurality of markers to one or both of the user and the hip brace; and
recording the walking motion of the user with a video camera configured to record data representing a movement of the markers during the walking motion.

18. The orthosis system of claim 17, further comprising the steps of:
processing the data representing the movement of the markers; and
storing the processed data.
